(19) United States
(12) Patent Application Publication
(10) Pub. No.: US 2016/0009697 A1
(43) Pub. Date: Jan. 14, 2016

(54) PYRIDINE DERIVATIVES AS 5-HT6 RECEPTOR ANTAGONISTS

(71) Applicant: Selvita S.A., Krakow (PL)

(72) Inventors: Charles-Henry FABRITIUS, Krakow (PL); Michal GALEZOWSKI, Skierniewice (PL); Marcin KROL, Krakow (PL); Mateusz NOWAK, Krakow (PL)

(21) Appl. No.: 14/769,289

(22) PCT Filed: Feb. 20, 2014
(86) PCT No.: PCT/EP2014/053344

§ 371 Date: Aug. 20, 2015

Related U.S. Application Data
(60) Provisional application No. 61/767,540, filed on Feb. 21, 2013.

(51) Int. Cl. C07D 405/14; A61K 31/496; A61K 31/4545; C07D 413/14

(52) U.S. Cl. CPC C07D 405/14; C07D 413/14; A61K 31/496; A61K 31/4545

(57) ABSTRACT

Compounds of formula (I), wherein X, Y, Z, R₁-R₇, m and n are as defined herein, exhibit 5-HT₆ antagonistic activity and are thus useful for the treatment of certain CNS disorders. Methods of use of said compounds are also provided.

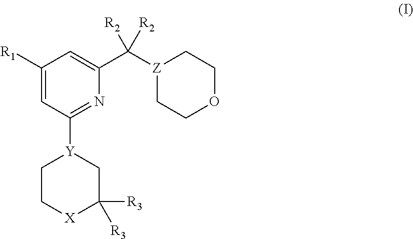

### TECHNICAL FIELD

The present disclosure relates to pharmacologically active pyridine derivatives, or pharmaceutically acceptable salts and esters thereof, as well as to pharmaceutical compositions comprising them and to their use as 5-hydroxytryptamine-6 (5-HT₆) receptor antagonists.

### BACKGROUND OF THE INVENTION

The 5-hydroxytryptamine-6 (5-HT₆) receptor is one of the most recently identified serotonin 5-HT receptors. In human and mouse the 5-HT₆ receptor is a 440-amino acid polypeptide with seven transmembrane spanning domains typical of the G-protein-coupled receptors. The extensive distribution of mRNA of 5-HT₆ in the brain has been reported based on northern blots. Highest levels of 5-HT₆ receptor mRNA have been observed in the striatum, the olfactory tubercle, hippocampus, nucleus accumbens, and dentate gyrus. There are also lower levels of 5-HT₆ receptor mRNA reported in the cortex, the amygdale, the granular layer of the cerebellum, and several diencephalic nuclei. There may be species differences in the expression pattern in the brain. It appears as though 5-HT₆ receptor mRNA is mainly present in the brain tissue, with little expression in peripheral tissues. The high affinity of a number of antipsychotic agents for the 5-HT₆ receptor, in addition to its mRNA localization, suggests that some of the clinical actions of those compounds may be mediated through this receptor. Therefore, 5-HT₆ receptor ligands are believed to be of potential use in the treatment of certain CNS disorders, such as anxiety, depression, epilepsy, obsessive compulsive disorder, attention deficit disorders, migraine, cognitive memory enhancement (for example, for the treatment of Alzheimer's disease), sleep disorders, feeding disorders (for example, anorexia or bulimia), neurodegenerative disorders (for example, head trauma or stroke), panic attacks, withdrawal from drug abuse (for example, withdrawal from cocaine, ethanol, nicotine, or benzodiazepines), schizophrenia, or the like; or in the treatment of certain gastrointestinal disorders, such as irritable bowel syndrome. Furthermore, the effect of 5-HT₆ antagonist, 5-HT₆ agonist, and 5-HT₆ antisense oligonucleotides to reduce food intake in rats has been reported.

WO2010/053388 discloses some 4,6-disubstituted 2-(4-methylpiperazin-1-yl)pyridine derivatives useful as monoaminergic receptor modulating agents. Paluchowska et al, Arch. Pharm. Pharm. Med. Chem. 2003, 2, 104-110 discloses series of 4,6-disubstituted 2-(1-piperazinyl)pyridines, the preparation of those compounds, and their 5-HT_{2A} receptor activity.

### SUMMARY OF THE INVENTION

An object of the present disclosure is to provide novel, potent and selective 5-HT₆ receptor antagonists that can be used for the treatment of disorders, conditions, or diseases mediated by the activity of 5-HT₆ receptors. Accordingly, an object of the present disclosure is to provide further compounds to be used as 5-HT₆ receptor antagonists in the treatment of mammals, including humans and animals. Furthermore, pharmaceutical compositions comprising the presently disclosed compounds are also provided.

The compounds of the present disclosure have an enhanced binding affinity for the 5-HT₆ receptor and/or an improved selectivity for the 5-HT₆ receptor, particularly over dopamine receptor D₂.

### DETAILED DESCRIPTION OF THE INVENTION

The present disclosure relates to novel pyridine derivatives having the general formula I,

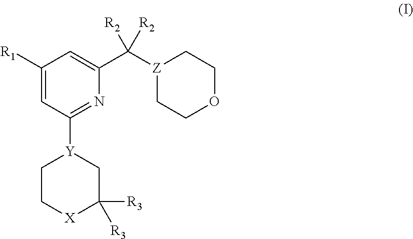

wherein

### X is NR₄ or CR₅H;

### Y is N or CH;

### Z is CH or N;

### R₁ is

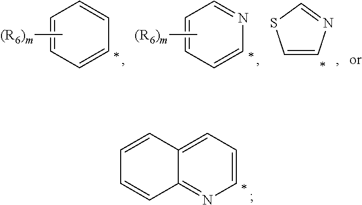

wherein the atom marked with the asterisk is bonded to the parent molecular moiety;
R₂ is, independently at each occurrence, H, (C₁-C₃)alkyl, or halogen;
or R₂ and R₂ both bonded to the same carbon atom form, together with the carbon atom to which they are bonded, a —(C═O) group;
R₃ is, independently at each occurrence, H, (C₁-C₃)alkyl, or halogen;
or R₃ and R₃ both bonded to the same carbon ring atom form, together with the carbon ring atom to which they are bonded, a —(C═O) group;
R₄ is H, (C₁-C₃)alkyl, or CF₃;

### R₅ is N(R₇)₂;

R₆ is, independently at each occurrence, hydroxy, halogen, (C₁-C₃)alkyl, (C₁-C₃)alkoxy, CF₃, or (C₁-C₃)alkoxy(C₁-C₃)alkoxy;
R₇ is, independently at each occurrence, H or (C₁-C₃)alkyl;
m is 0, 1, 2, or 3; and
n is 0, 1, or 2
or a pharmaceutically acceptable salt or ester thereof.

In at least one embodiment,

### X is NR₄ or CR₅H;

### Y is N;

### Z is CH;

### R₁ is

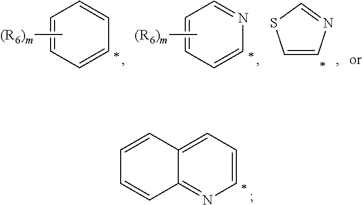

wherein the atom marked with the asterisk is bonded to the parent molecular moiety;
R₂ is, independently at each occurrence, H or (C₁-C₃)alkyl;
R₃ is, independently at each occurrence, H or (C₁-C₃)alkyl;
R₄ is H or (C₁-C₃)alkyl:

### R₅ is N(R₇)₂;

R₆ is, independently at each occurrence, halogen or (C₁-C₃)alkoxy;

### R₇ is H;

m is 1 or 2; and
n is 0.

In another embodiment,

### X is NR₄;

### Y is N;

### Z is CH;

### R₁ is

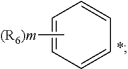

wherein the atom marked with the asterisk is bonded to the parent molecular moiety;
R₂ is, independently at each occurrence, H or (C₁-C₃)alkyl;
R₃ is, independently at each occurrence, H or (C₁-C₃)alkyl;
R₄ is H or (C₁-C₃)alkyl:
R₆ is halogen; and
m is 1.

In another embodiment,

### X is NR₄;

### Y is N;

### Z is CH;

### R₁ is

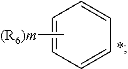

wherein the atom marked with the asterisk is bonded to the parent molecular moiety;
R₂ is, independently at each occurrence, H or (C₁-C₃)alkyl;
R₃ is, independently at each occurrence, H or (C₁-C₃)alkyl;
R₄ is H or (C₁-C₃)alkyl:
R₆ is (C₁-C₃)alkoxy; and
m is 1.

In further embodiments, the compounds of formula I are 1-[4-(4-methoxyphenyl)-6-(oxan-4-ylmethyl)pyridin-2-yl]-4-methylpiperazine, 2-[2-(4-methylpiperazin-1-yl)-6-(oxan-4-ylmethyl)pyridin-4-yl]quino line, 1-methyl-4-[6-(oxan-4-ylmethyl)-4-(pyridin-2-yl)pyridin-2-yl]piperazine, 1-methyl-4-[6-(oxan-4-ylmethyl)-4-(1,3-thiazol-4-yl)pyridin-2-yl]-piperazine, 1-[4-(2-fluoro-4-methoxyphenyl)-6-(oxan-4-ylmethyl)pyridin-2-yl]-4-methylpiperazine, 1-[4-(4-methoxyphenyl)-6-(oxan-4-ylmethyl)pyridin-2-yl]piperidin-4-amine, 1-[4-(4-methoxyphenyl)-6-[1-(oxan-4-yl)ethyl]pyridin-2-yl]-4-methylpiperazine, 1-[4-(4-methoxyphenyl)-6-(oxan-4-ylmethyl)pyridin-2-yl]-3,3-dimethylpiperazine, 1-[4-(4-methoxyphenyl)-6-(oxan-4-ylmethyl)pyridin-2-yl]piperazine, 1-[4-(4-methoxyphenyl)-6-(oxan-4-ylmethyl)pyridin-2-yl]-4-methyl-piperazine maleate, 1-[4-(4-methoxyphenyl)-6-(oxan-4-ylmethyl)pyridin-2-yl]-4-methyl-piperazine tartrate, 1-[4-(4-methoxyphenyl)-6-(oxan-4-ylmethyl)pyridin-2-yl]-4-methyl-piperazine citrate, or 1-[4-(4-methoxyphenyl)-6-(oxan-4-ylmethyl)pyridin-2-yl]-4-methyl-piperazine fumarate.

The terms employed herein have the meanings indicated below.

The term “halo” or “halogen,” as employed herein as such or as part of another group, refers to fluorine, chlorine, bromine, or iodine.

The term “(C₁-C₃)alkyl,” as employed herein as such or as part of another group, refers to a saturated hydrocarbon group having a straight or branched chain, containing 1, 2, or 3 carbon atom(s). Representative examples of (C₁-C₃)alkyl include, but are not limited to, methyl, ethyl, n-propyl, and isopropyl.

The term “(C₁-C₃)alkoxy,” as employed herein as such or as part of another group, refers to an (C₁-C₃)alkyl group, as defined herein, appended to the parent molecular moiety through an oxygen atom. Representative examples of (C₁-C₃)alkoxy include, but are not limited to, methoxy, ethoxy, and n-propoxy.

The term “(C₁-C₃)alkoxy(C₁-C₃)alkoxy,” as employed herein as such or as part of another group, refers to at least one (C₁-C₃)alkoxy group, as defined herein, appended to the parent molecular moiety through an (C₁-C₃)alkoxy group, as defined herein. When there are several (C₁-C₃)alkoxy groups, the (C₁-C₃)alkoxy groups can be attached to the same or different carbon atom and the (C₁-C₃)alkoxy groups can be identical or different. Representative examples of (C₁-C₃)alkoxy(C₁-C₃)alkoxy include, but are not limited to, methoxymethoxy, propoxymethoxy, 2-methoxyethoxy, 2-ethoxyethoxy, 2,2-dimethoxyethoxy, 1-methyl-2-propoxyethoxy, and 2-methoxypropoxy.

The expression “compounds of the present disclosure” as employed herein refers to the compounds of formula I.

The terms “same carbon atom” and “same carbon ring atom” as used herein refer to a carbon atom in Formula I to which two defined substituents are bonded, i.e. either R₂ or R₃.

The “pharmaceutically acceptable salts” according to the present disclosure include therapeutically active, non-toxic, base and and acid salt forms, which the compounds of formula I are able to form with both organic and inorganic bases and acids. Representative examples of pharmaceutically acceptable base addition salt forms, for example, metal or amine salts, include, but are not limited to, ammonium salts, lithium, sodium, potassium, calcium, magnesium, aluminum, and zinc salts, salts with organic bases, such as N-methyl-D-glucamine, hydrabamine salts, and salts with amino acids, such as arginine, lysine, and the like. Representative examples of pharmaceutically acceptable acid addition salts include, but are not limited to, chlorides, bromides, sulfates, nitrates, phosphates, sulfonates, methane sulfonates, formates, tartrates, maleates, citrates, benzoates, salicylates, ascorbates, acetates and oxalates, fumarates, and succinates.

Pharmaceutically acceptable esters, when applicable, may be prepared by known methods using pharmaceutically acceptable acids that are conventional in the field of pharmaceuticals and that retain the pharmacological properties of the free form. Non-limiting examples of these esters include esters of aliphatic or aromatic alcohols. Representative examples of pharmaceutically acceptable esters include, but are not limited to, methyl, ethyl, n-propyl, isopropyl, n-butyl, isobutyl, sec-butyl, tert-butyl, and benzyl esters.

The present disclosure includes all the possible geometric isomers, for example Z and E isomers (cis and trans isomers), of the compounds, as well as all the possible optical isomers, such as diastereomers and enantiomers, of the compounds. Furthermore, the present disclosure includes both the individual isomers and any mixtures thereof, such as a racemic mixture. The individual isomers may be obtained using the corresponding isomeric forms of the starting material or they may be separated after the preparation of the end compound according to conventional separation methods. For the separation of optical isomers, such as enantiomers, from the mixture thereof, conventional resolution methods, for example, fractional crystallization or preparative chiral chromatography, may be used.

The compounds of formula I can be prepared by a variety of synthetic routes analogously to, or according to methods known in the literature using suitable starting materials. The starting materials depicted below are commercially available or can be prepared via synthetic routes known in the literature.

In general, compounds of formula I can be prepared analogoulsly or according to scheme 1:

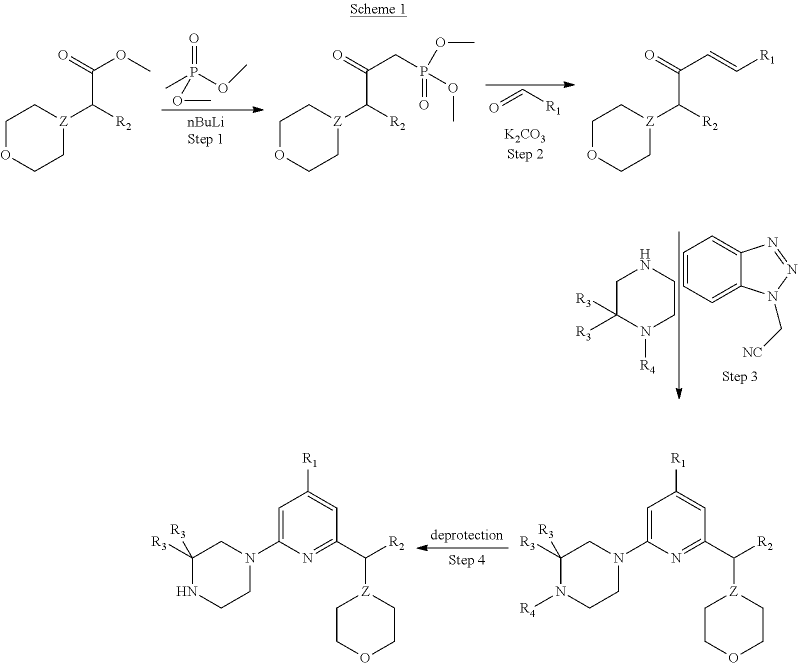

Any starting material or intermediate in the reactions to prepare compounds according to the present disclosure can be protected, if necessary, in a manner known in the art. Any protected functionality can subsequently be deprotected in a manner known in the art.

The synthetic route described above is meant to illustrate the preparation of the compounds of formula I, and the preparation is not limited thereto, that is, there are also other possible synthetic methods which are within the general knowledge of a person skilled in the art.

The compounds of formula I may be converted, if desired, into their pharmaceutically acceptable salt or ester forms using methods known in the art.

The present disclosure will be explained in more detail by the following examples.

The examples are meant for illustrating purposes only and do not limit the scope of the invention defined in the claims. It is noted that examples 1 to 9 have been prepared as an hydrochloride or fumaric salt and structural characterization by NMR has been done on the salt form.

Normal phase flash chromatography purifications were performed using Interchim instruments together with disposable Interchim columns (50 μm). Preparative HPLC purifications were done with a Shimadzu LC-20AP preparative HPLC system equipped with Gemini-NX C18 (150×4.6 mm) column. Gradient of water/acetonitrile with 0.05% oftrifluoroacetic acid was used as eluent. Chiral preparative HPLC were done with Agilent 1200 equipped with Chiralpak IA (250×20 mm). Gradient of n-hexane/iPrOH with 0.2% of DEA was used as eluent. The chemical purity of the products were confirmed by Bruker Amazon/Dionex LC-MS system equipped with a Waters C18 (1.8 μm, 2.1×50 mm and 5 μm, 3.9×150 mm) columns. A gradient of water/acetonitrile with 0.1% of formic acid was used as eluent. The chiral purity of the products were confirmed by Agilent 1100 LC-MS system equipped with a Chiralpak IA column. A gradient of n-hexane/iPrOH with 0.1% of DEA was used as eluent. The structures of the products were confirmed by ¹H NMR. The spectra were measured with Varian Mercury-VX 300 MHz. The following general abbreviations are used: iPrOH=isopropanol, DEA=diethanolamine, THF=tetrahydrofuran, DMSO-d₆=dimethyl sulfoxide-d₆, TLC=thin layer chromatography.

### PREPARATION OF THE COMPOUNDS OF THE PRESENT DISCLOSURE

### Example 1

### 1-[4-(4-Methoxyphenyl)-6-(oxan-4-ylmethyl)pyridin-2-yl]-4-methyl-piperazine

### Step 1: Dimethyl[3-(oxan-4-yl)-2-oxopropyl]phosphonate

Dimethyl methylphosphonate (48.5 mmol, 6.02 g) was dissolved in dry THF (40 ml) and the solution was cooled to −78° C. A solution of n-butyllithium (47.9 mmol, 3.1 g, 1.6 M in hexane) was added dropwise under Ar. After 1 hour the solution of methyl 2-(tetrahydro-2H-pyran-4-yl)acetate (12.1 mmol, 1.92 g) in dry THF (10 ml) was added and the resulting mixture was stirred at −78° C. for 1.5 hour. The mixture was quenched with saturated aqueous ammonium chloride and allowed to reach room temperature. The two layers were separated and aqueous phase was extracted with dichloromethane. Organic layers were combined, extracted with saturated brine, dried over magnesium sulphate and concentrated in vacuum. 3.0 g of the title compound was obtained as a yellow oil. The crude product was used as such.

### Step 2: 4-(4-Methoxyphenyl)-1-(oxan-4-yl)but-3-en-2-one

Dimethyl [3-(oxan-4-yl)-2-oxopropyl]phosphonate (8.0 mmol, 2.0 g) and potassium carbonate (24.0 mmol, 3.31 g) were dissolved in ethanol (50 ml) and stirred for 1 hour. 4-anisaldehyde (8.0 mmol, 1.09 g) was added and the resulting mixture was stirred overnight at room temperature. The solvent was evaporated and the residue was dissolved in dichloromethane and washed twice with water and once with brine. Organic layer was dried over magnesium sulphate and evaporated under vacuum. The product was purified by SiO₂ column chromatography (1% of methanol in dichloromethane) to give 1.7 g of product as a white solid.

### Step 3: 1-[4-(4-Methoxyphenyl)-6-(oxan-4-ylmethyl)pyridin-2-yl]-4-methylpiperazine

4-(4-Methoxyphenyl)-1-(oxan-4-yl)but-3-en-2-one (6.5 mmol, 1.7 g), 1H-benzotriazole-1-acetonitrile (6.5 mmol, 1.0 g), N-methylpiperazine (38.9 mmol, 3.9 g) were placed in a seal-tube and dissolved in ethanol (20 ml). Molecular sieves were added and the resulting mixture was stirred at 90° C. overnight. 1H-benzotriazole-1-acetonitrile (6.5 mmol, 1.03 g) was added to the mixture and reaction was continued at 90° C. overnight. Progress of reaction was monitored by TLC and 4-(4-methoxyphenyl)-1-(oxan-4-yl)but-3-en-2-one was still observed. After another portion of 1H-benzotriazole-1-acetonitrile (6.5 mmol, 1.03 g) and stirring overnight at 90° C., molecular sieves were filtered off and ethanol was evaporated with vacuum. The crude residue was dissolved in dichloromethane and washed three times with sodium hydroxide (10% solution in water), saturated aqueous sodium bicarbonate and saturated brine. The organic layer was dried over magnesium sulphate and evaporated with vacuum. Final product was separated by column chromatography (5% methanol in ethyl acetate) to give 1.5 g of the title compound as yellow oil.

¹H NMR (600 MHz, DMSO-d₆): δ 11.39 (s, 1H), 7.89 (s, 2H), 7.30-7.02 (m, 4H), 4.61 (d, 2H), 3.85-3.80 (m, 5H), 3.52 (d, 2H), 3.25 (td, 2H), 3.16 (d, 2H), 2.81 (m, 5H), 2.03 (m, 1H), 1.53 (d, 2H), 1.30 (qd, 2H).

### Example 2

### 2-[2-(4-Methylpiperazin-1-yl)-6-(oxan-4-ylmethyl)pyridin-4-yl]quinoline

The synthesis was done using the same method as in Example 1 except that 4-anisaldehyde (Step 2) was replaced with 2-quinolinecarbaldehyde. 66 mg of the title product was obtained as a yellow solid.

¹H NMR (600 MHz, DMSO-d₆): δ 11.28 (s, 1H), 8.62 (d, 1H), 8.35 (d, 1H), 8.19 (d, 1H), 8.08 (d, 1H), 7.87 (t, 1H), 7.70 (t, 2H), 7.58 (s, 1H), 4.63 (d, 2H), 3.83 (d, 2H), 3.55 (d, 2H), 3.47 (s, 2H), 3.28 (t, 2H), 3.17 (d, 2H), 2.82 (s, 3H), 2.06 (m, 1H), 1.57 (d, 2H), 1.32 (d, 2H).

### Example 3

### 1-Methyl-4-[6-(oxan-4-ylmethyl)-4-(pyridin-2-yl)pyridin-2-yl]piperazine

The synthesis was done using the same method as in Example 1 except that 4-anisaldehyde (Step 2) was replaced with pyridine-2-carbaldehyde. 76 mg of the title product was obtained as a yellow solid.

¹H NMR (300 MHz, DMSO-d₆): δ 8.67 (d, 1H), 8.03 (d, 1H), 7.89 (td, 1H), 7.41 (dd, 1H), 7.25 (s, 1H), 7.17 (s, 1H), 6.58 (s, 2H), 3.79 (d, 2H), 3.57 (t, 4H), 3.24 (t, 2H), 2.59-2.50 (m, 6H), 2.29 (s, 3H), 1.96 (m, 1H), 1.51 (d, 2H), 1.28 (dd, 2H).

### Example 4

### 1-Methyl-4-[6-(oxan-4-ylmethyl)-4-(1,3-thiazol-4-yl)pyridin-2-yl]-piperazine

The synthesis was done using the same method as in Example 1 except that 4-anisaldehyde (Step 2) was replaced with 1,3-thiazole-4-carbaldehyde. 75 mg of the title product was obtained as a white solid.

¹H NMR (600 MHz, CDCl₃): δ 8.88 (d, 1H), 7.66 (d, 1H), 7.08 (d, 1H), 6.92 (d, 1H), 3.94 (dd, 2H), 3.65 (t, 4H), 3.38 (td, 2H), 2.62 (d, 2H), 2.56 (t, 4H), 2.36 (s, 3H), 2.07 (m, 1H), 1.62 (dd, 2H), 1.40 (ddd, 2H).

### Example 5

### 1-[4-(2-Fluoro-4-methoxyphenyl)-6-(oxan-4-ylmethyl)pyridin-2-yl]-4-methylpiperazine

The synthesis was done using the same method as in Example 1 except that 4-anisaldehyde (Step 2) was replaced with 2-fluoro-4-methoxy benzaldehyde. 90 mg of the title product was obtained as a light yellow solid.

¹H NMR (300 MHz, DMSO-d₆): δ 7.49 (t, 1H), 6.89 (m, 2H), 6.68 (s, 1H), 6.61 (s, 1H), 6.58 (s, 2H), 3.83-3.74 (m, 5H), 3.52 (t, 4H), 3.23 (td, 2H), 2.51 (dd, 5H), 2.47 (s, 1H), 2.27 (s, 3H), 1.99-1.87 (m, 1H), 1.50 (d, 2H), 1.29-1.14 (m, 2H).

### Example 6

### 1-[4-(4-Methoxyphenyl)-6-(oxan-4-ylmethyl)pyridin-2-yl]piperidin-4-amine

The synthesis was done using the same method as in Example 1 except that N-methylpiperazine (Step 3) was replaced with tert-butyl N-(piperidin-4-yl)carbamate. Deprotection of the Boc group was done according to a procedure described in the book of Protective Groups in Organic Synthesis and gave 25 mg of the title product as a yellow solid.

¹H NMR (600 MHz, DMSO-d₆): δ 7.71-7.67 (m, 2H), 7.04-7.00 (m, 2H), 6.85 (s, 1H), 6.75 (d, 1H), 6.48 (s, 2H), 4.43 (d, 2H), 3.81 (m, 5H), 3.26 (t, 4H), 2.88 (t, 2H), 2.54 (d, 2H), 1.94 (m, 3H), 1.52 (m, 4H), 1.32-1.20 (m, 3H).

### Example 7

### 1-[4-(4-Methoxyphenyl)-6-[1-(oxan-4-yl)ethyl]pyridin-2-yl]-4-methyl-piperazine

The synthesis was done using the same method as in Example 1 except that methyl 2-(tetrahydro-2H-pyran-4-yl)acetate (Step 1) was replaced with methyl 2-(oxan-4-yl)propanoate which was synthesized according to a procedure described in EP1431285 A1. 95 mg of the title product was obtained as a white solid.

¹H NMR (300 MHz, DMSO-d₆): δ 7.68 (d, 2H), 7.00 (d, 2H), 6.78 (s, 1H), 6.72 (s, 1H), 6.58 (s, 2H), 3.84 (dd, 2H), 3.78 (s, 3H), 3.74 (m, 1H), 3.71 (m, 1H), 3.54 (t, 4H), 3.30-3.09 (m, 7H), 2.51 (d, 1H), 2.26 (s, 3H), 1.69 (d, 1H), 1.17 (d, 4H).

### Example 8

### 1-[4-(4-Methoxyphenyl)-6-(oxan-4-ylmethyl)pyridin-2-yl]-3,3-dimethylpiperazine

### Step 1: 2,6-Dichloro-4-(tetramethyl-1,3,2-dioxaborolan-2-yl)pyridine

2,6-Dichloropyridine (20.3 mmol, 3.0 g), bis-(pinacolato)diboron (22.3 mmol, 5.66 g) and 1,10-phenantroline (1.2 mmol, 0.212 g) were dissolved in dry 1,2-dichloroethane (100 ml), degassed and purged with Ar for 5 minutes. Chloro-1,5-cyclooctadiene iridium(I) dimer (0.445 g, 0.7 mmol) was added and the resulting reaction mixture was stirred at 100° C. for 15 hours in sealed tube conditions. After completion of the reaction the contents were cooled, filtered through Celite and 1,2-dichloroethane was evaporated with vacuum. The crude residue was dissolved in diethyl ether and washed with sodium hydroxide (4N solution in water). The aqueous layer was acidified to pH=1 with HCl (6N solution in water) and resulting solid was filtered off and washed with water. 4.8 g of the title compound was obtained as a grey solid.

### Step 2: 2,6-Dichloro-4-(4-methoxyphenyl)pyridine

2,6-Dichloro-4-(tetramethyl-1,3,2-dioxaborolan-2-yl)pyridine (10.95 mmol, 3.0 g), 1-bromo-4-methoxybenzene (21.9 mmol, 4.09 g) and Bis(triphenylphosphine)palladium(II) dichloride (1.1 mmol, 0.879 g) were dissolved in 1,4-dioxane (165 ml) and saturated sodium carbonate (25 ml). The resulting reaction mixture was stirred at 100° C. for 15 hours in sealed tube conditions. After completion of the reaction the contents were cooled and concentrated with vacuum. The crude residue was dissolved in ethyl acetate and washed with saturated brine. Organic layer was separated, dried over magnesium sulphate and concentrated in vacuum. Final product was separated by Flash column chromatography (hexane/ethyl acetate) to give 3.0 g of the title compound.

### Step 3: 1-[6-Chloro-4-(4-methoxyphenyl)pyridin-2-yl]-3,3-dimethylpiperazine

2,6-Dichloro-4-(4-methoxyphenyl)pyridine (0.79 mmol, 0.200 g), 2,2-dimethylpiperazine (0.79 mmol, 0.090 g) and Hunig's base (0.9 mmol, 0.108 g) were dissolved in 1,4-dioxane (3 ml) and the resulting reaction mixture was stirred at 120° C. for 18 hours in sealed tube conditions. After completion of the reaction the contents were cooled and 1,4-dioxane was evaporated with vacuum. The crude residue was dissolved in DCM and washed with saturated sodium bicarbonate.

Organic layer was separated, dried over sodium sulphate and concentrated in vacuum. Final product was separated by Flash column chromatography (DCM/methanol) to give 0.08 g of the title compound.

### Step 4: 1-[4-(4-Methoxyphenyl)-6-(oxan-4-ylidenemethyl)pyridin-2-yl]-3,3-dimethylpiperazine

### Synthesis of 4-methyl-N′-(oxan-4-ylidenemethyl)benzene-1-sulfonohydrazide

To a rapidly stirred solution of 4-methylbenzene-1-sulfonohydrazide (0.13 mmol, 2.5 g) in methanol (5 ml), oxane-4-carbaldehyde (0.13 mmol, 1.48 g) was added dropwise. The reaction mixture was stirred at room temperature for 30 minutes and then cooled to 0° C. 3.2 g of title product was filtered off as white solid. 1-[6-Chloro-4-(4-methoxyphenyl)pyridin-2-yl]-3,3-dimethylpiperazine (0.24 mmol, 0.080 g) 2-Dicyclohexylphosphino-2′,4′,6′-triisopropylbiphenyl (0.009 g), 4-methyl-N′-(oxan-4-ylidenemethyl)benzene-1-sulfonohydrazide (0.3 mmol, 0.075 g) and lithium tert-butoxide (0.5 mmol, 0.044 g) were dissolved in dry 1,4-dioxane (1.5 ml), degassed and purged with Ar for 5 minutes.

Tris(dibenzylideneacetone)dipalladium(0) (0.004 g) was added and the mixture was degassed and purged with Ar again. The resulting reaction mixture was stirred at 110° C. over 2 days in sealed tube conditions. After completion of the reaction the contents were cooled, filtered through Celite and 1,4-dioxane was evaporated with vacuum. The crude residue was dissolved in dichloromethane and washed with water. Organic layer was separated, dried over sodium sulphate and concentrated in vacuum. Final product was separated by Flash column chromatography (DCM/methanol) to give 0.06 g of the title compound.

### Step 5: 1-[4-(4-Methoxyphenyl)-6-(oxan-4-ylmethyl)pyridin-2-yl]-3,3-dimethylpiperazine

1-[4-(4-Methoxyphenyl)-6-(oxan-4-ylidenemethyl)pyridin-2-yl]-3,3-dimethylpiperazine (0.13 mmol, 0.05 g) was dissolved in methanol (3 ml). The resulting mixture was degassed and purged with Ar for 5 minutes and then 10% palladium on charcoal (0.1 eq.) was added. The resulting mixture was stirred overnight under hydrogen atmosphere (ambient pressure). The reaction mixture was filtered through Celite and the filtrate was concentrated in vacuum. Final product was separated by Flash column chromatography (DCM/methanol/1% Et₃N) to give 0.012 g of the title compound as a white solid.

¹H NMR (300 MHz, DMSO-d₆): δ 7.69 (d, 2H), 7.01 (d, 2H), 6.86 (s, 1H), 6.76 (s, 1H), 6.48 (s, 2H), 3.79 (m, 5H), 3.73-3.63 (m, 2H), 3.53 (s, 3H), 3.29-3.20 (m, 4H), 3.11-3.02 (m, 2H), 2.52 (d, 2H), 1.95 (m, 1H), 1.55-1.44 (m, 2H), 1.24 (m, 8H).

### Example 9

### 1-[4-(4-Methoxyphenyl)-6-(oxan-4-ylmethyl)pyridin-2-yl]piperazine

The synthesis was done using the same method as in Example 1 except that N-methylpiperazine (Step 3) was replaced with 1-benzylpiperazine. N-debenzylation of 1-benzyl-4-[4-(4-methoxyphenyl)-6-(oxan-4-ylmethyl)pyridin-2-yl]piperazine was done according to a procedure described in WO2008/25736 A1. 1.0 g of the title product was obtained as a white solid.

¹H NMR (300 MHz, DMSO-d₆): δ 7.69 (d, 2H), 7.01 (d, 2H), 6.86 (s, 1H), 6.81 (s, 1H), 6.48 (s, 2H), 3.79 (m, 5H), 3.66 (t, 4H), 3.29-3.18 (m, 2H), 3.04 (t, 4H), 2.53 (d, 2H), 1.95 (m, 1H), 1.50 (d, 2H), 1.23 (m, 2H).

### Example 10

### 1-[4-(4-Methoxyphenyl)-6-(oxan-4-ylmethyl)pyridin-2-yl]-4-methyl-piperazine maleate

1-[4-(4-Methoxyphenyl)-6-(oxan-4-ylmethyl)pyridin-2-yl]-4-methylpiperazine free base (0.66 mmol, 250 mg) was dissolved in isopropyl alcohol (10 ml) and stirred at 70° C. for 15 minutes. Maleic acid (0.66 mmol, 76.1 mg) was added to the solution and stirred at 70° C. for 2 hours. The resulting mixture was allowed to get at room temperature and then put into the fridge. After 16 h maleic acid salt was filtered off and washed with isopropyl alcohol. 226 mg of the product was obtained as a white solid.

¹H NMR (300 MHz, DMSO-d₆): 7.71 (d, 2H), 7.02 (d, 2H), 6.94 (s, 1H), 6.86 (d, 1H), 6.02 (s, 2H), 3.79 (s, 5H), 3.23 (td, 4H), 2.81 (s, 3H), 2.55 (d, 2H), 1.95 (m, 1H), 1.50 (d, 2H), 1.25 (qd, 2H).

### Example 11

### 1-[4-(4-Methoxyphenyl)-6-(oxan-4-ylmethyl)pyridin-2-yl]-4-methyl-piperazine tartrate

1-[4-(4-Methoxyphenyl)-6-(oxan-4-ylmethyl)pyridin-2-yl]-4-methylpiperazine free base (0.66 mmol, 250 mg) was dissolved in isopropyl alcohol (10 ml) and stirred at 70° C. for 15 minutes. Tartaric acid (0.66 mmol, 98.4 mg) was added to the solution and stirred at 70° C. for 2 hours. The resulting mixture was allowed to get at room temperature and then put into the fridge. After 16 h, tartaric acid salt was filtered off and washed with isopropyl alcohol. 250 mg of the product was obtained as a white solid.

¹H NMR (300 MHz, DMSO-d₆): 7.71-7.65 (m, 2H), 7.00 (d, 2H), 6.79 (d, 2H), 4.15 (s, 2H), 3.78 (m, 5H), 3.58 (s, 4H), 3.23 (td, 2H), 2.61 (s, 4H), 2.53 (d, 2H), 2.36 (s, 3H), 1.94 (m, 1H), 1.50 (d, 2H), 1.22 (qd, 2H).

### Example 12

### 1-[4-(4-Methoxyphenyl)-6-(oxan-4-ylmethyl)pyridin-2-yl]-4-methyl-piperazine citrate

1-[4-(4-Methoxyphenyl)-6-(oxan-4-ylmethyl)pyridin-2-yl]-4-methylpiperazine free base (0.66 mmol, 250 mg) was dissolved in isopropyl alcohol (10 ml) and stirred at 70° C. for 15 minutes. Citric acid (0.66 mmol, 125.9 mg) was added to the solution and stirred at 70° C. for 2 hours. The resulting mixture was allowed to get at room temperature and then put into the fridge. After 16 h citric acid salt was filtered off and washed with isopropyl alcohol. 295 mg of the product was obtained as a white solid.

¹H NMR (300 MHz, DMSO-d₆): 7.69 (d, 2H), 7.01 (d, 2H), 6.83 (d, 2H), 3.79 (m, 5H), 3.66 (s, 4H), 3.23 (t, 2H), 2.85 (s, 4H), 2.63 (d, 2H), 2.56 (s, 2H), 2.53 (m, 4H), 1.95 (m, 1H), 1.50 (d, 2H), 1.23 (qd, 2H).

### Example 13

### 1-[4-(4-Methoxyphenyl)-6-(oxan-4-ylmethyl)pyridin-2-yl]-4-methyl-piperazine fumarate

1-[4-(4-Methoxyphenyl)-6-(oxan-4-ylmethyl)pyridin-2-yl]-4-methylpiperazine free base (0.66 mmol, 250 mg) was dissolved in isopropyl alcohol (10 ml) and stirred at 80° C. for 15 minutes. Fumaric acid (0.66 mmol, 76.1 mg) was added to the solution and stirred at 80° C. for 2 hours. The resulting mixture was allowed to get at room temperature and then put into the fridge. After 30 minutes, fumaric acid salt was filtered off and washed with isopropyl alcohol. 213 mg of the product was obtained as a white solid.

¹H NMR (300 MHz, DMSO-d₆): 7.67 (dd, 2H), 7.00 (dd, 2H), 6.77 (d, 2H), 6.58 (s, 2H), 3.78 (m, 5H), 3.53 (t, 4H), 3.25 (t, 4H), 2.53 (s, 4H), 2.25 (s, 3H), 1.94 (m, 1H), 1.50 (dd, 2H), 1.22 (dq, 2H).

As already mentioned hereinbefore, the compounds of formula I show interesting pharmacological properties, namely they exhibit an improved selectivity for the 5-HT₆ receptors and/or enhanced potency over the other known receptors and drug targets. Said properties are demonstrated, for example, with the pharmacological tests presented below.

### Experiment 1: 5-HT₆ Binding Affinity

Compounds of the present disclosure were evaluated using transfected cell types receptive specifically to the 5-HT₆ receptor. The assay protocol generally entailed the incubation of membranes prepared from cells expressing the 5-HT₆ receptor with [³H]-LSD (2 nM). Increasing levels of the test compound were incubated with the radioligand and the membrane homogenates prepared from the recombinant cells. After a 60 minute incubation at 37° C., the incubation was terminated by vacuum filtration. The filters were washed with buffer and the filters were counted for radioactivity using liquid scintillation spectrometry. Concentrations ranging from 10⁻¹² M to 10⁻⁵ M of the test compound were evaluated. For comparison, the affinity of SB271046 (Ki=0.5 nM±0.2) for the 5-HT₆ receptor was used as a standard. Binding in the presence of varying concentrations of test compound is expressed as a percentage of specific binding in the absence of test compound. The results are plotted as log % bound versus log concentration of test compound. Nonlinear regression analysis of data points with a computer assisted program Prisms yielded both the IC50 and the Ki values of test compounds with 95% confidence limits. A linear regression line of data points is plotted, from which the IC50 value is determined and the Ki value is determined based upon the following equation:

Ki=IC50/(1+L/K_{D})

It has been shown that compounds of formula I have a good affinity to the 5-HT₆ receptor. The results are shown in Table 1.

TABLE 1
5-HT₆ equilibrium dissociation constants determined
in a competitive radioligand binding study

| Compound of example | 5-HT₆ binding Ki [nM] |
|---|---|
| 1 | 10.52 |
| 2 | 39.59 |
| 3 | 22.22 |
| 4 | 14.29 |
| 5 | 22.84 |
| 6 | 38.75 |
| 7 | 12.20 |
| 8 | 36.10 |
| 9 | 13.20 |

### Experiment 2: Dopamine Receptor D₂ Binding Affinity

Dopamine receptor D₂ binding affinity of compounds of formula I was also evaluated. The assay protocol generally entailed the incubation of membranes prepared from cells expressing the D₂ receptor with [³H]methyl-spiperone (0.2 nM). Increasing levels of the test compound were incubated with the radioligand and the membrane homogenates prepared from the recombinant cells. After a 60 minute incubation at 37° C., the incubation was terminated by vacuum filtration. The filters were washed with buffer and the filters were counted for radioactivity using liquid scintillation spectrometry. Concentrations ranging from 10⁻¹² M to 10⁻⁵ M of the test compound were evaluated. For comparison, the affinity of sulpiride (Ki=27.0±12.0) for the 5-HT₆ receptor was used as a standard. Binding in the presence of varying concentrations of test compound is expressed as a percentage of specific binding in the absence of test compound. The results are plotted as log % bound versus log concentration of test compound. Nonlinear regression analysis of data points with a computer assisted program Prisms yielded both the IC50 and the Ki values of test compounds with 95% confidence limits. A linear regression line of data points is plotted, from which the IC50 value is determined and the Ki value is determined based upon the following equation:

Ki=IC50/(1+L/KD)

The results are shown in Table 2.

TABLE 2
D₂ equilibrium dissociation constants determined
in a competitive radioligand binding study

| Compound of example | D₂ binding Ki [nM] |
|---|---|
| 1 | 107.10 |
| 2 | 4105.00 |
| 3 | 1184.00 |
| 4 | >10000.00 |
| 5 | 1247.00 |
| 6 | >10000.00 |
| 7 | >10000.00 |
| 8 | >10000.00 |
| 9 | >10000.00 |

### Experiment 3: 5-HT₆ Functional Assay

The serotonin receptor 5-HT₆ is a Gs coupled receptor. In the brain it responds to serotonin and other agonists by increasing adenyl cyclase mediated production of cyclic AMP. 5-HT₆ can be also functionally tested using different coupling types. Millipore's cloned human 5-HT₆-expressing cell line is made in the Chem-10 host, which supports high levels of recombinant 5-HT₆ expression on the cell surface and contains optimized levels of a recombinant promiscuous G protein to couple the receptor to the calcium signaling pathway. Cells were thawed, resuspended in media, dispensed into 96-well assay plates at a density of 27500 cells per well and, following over night recovery, assayed for calcium response. Tests were performed using Fluo-4NW Calcium Assay Kit (Molecular Probes #F36206) and standard testing procedure for this kit. Cells were preincubated with test compound for 30 min, then endogenous agonists serotonin was added. Fluorescence was measured by FLEXstation 3 (Molecular Devices). The apparent dissociation constants (Kb) were calculated by means of GraphPhad Prism 5 software using the modified Cheng Prusoff equation (Kb=IC50/(1+(A/EC50A)), where A=concentration of reference agonist in the assay and EC50A=EC50 value of the reference agonist). For each IC50 determination, agonist dose response curve is run in parallel with the antagonist, on the same experimental plate. Curves maximal and minimal values should be reached in assays and it is checked if Prism uses proper maximal and minimal values for curve fitting. The results are shown in Table 3.

TABLE 3
5-HT₆ antagonistic equilibrium dissociation
constants determined by functional assay

| Compound of example | 5-HT₆ antagonist Kb [nM] |
|---|---|
| 1 | 1.06 |
| 2 | 203.90 |
| 3 | 81.83 |
| 4 | 1.97 |
| 5 | 5.06 |
| 6 | >1000.00 |
| 7 | 0.01 |
| 8 | 4.70 |
| 9 | 1.64 |

### Experiment 4: D₂ Functional Assay

Dopamine D₂ receptor inhibits adenylyl cyclase activity. CHO cells (Chinese Hamster Ovary cell-line) transfected with short form of human D₂ receptor were used for D₂ functional testing. Cells were seeded at a density of 10 000 cells/well in white-walled 96-well plates one day before the experiment. Forskolin at 10 μM was used to increase the intracellular level of cAMP. Compounds were tested according to the procedure from cAMP-Glo™ kit manufacturer's instruction. The results are shown in Table 4.

TABLE 4
D₂ antagonistic equilibrium dissociation
constants determined by functional assay

| Compound of example | D₂ antagonist Kb [nM] |
|---|---|
| 1 | 537.50 |
| 2 | >3500.00 |
| 3 | >2000.00 |
| 4 | 781.00 |
| 5 | >2000.00 |
| 6 | >3500.00 |
| 7 | 339.60 |
| 8 | >3500.00 |
| 9 | >2000.00 |

### Experiment 5: H₁ Functional Assay

The histamine receptor H₁ is linked to an intracellular G-protein (Gq) that activates phospholipase C and the phosphatidylinositol (PIP2) signaling pathway. The pathway starts with stimulation of the receptor and further activation of phospholipase C (PLC). Inositol-1,4,5-triphosphate (IP3), which is one of the products of PLC activity, binds to IP3 receptor in smooth ER. Opening of IP3R, which acts as a calcium ion channel, causes an increase in Ca²⁺ concentration in the cytosol, which can be quantitatively measured using calcium-sensitive fluorescent dyes.

H₁ antagonism was measured in CHO cells (Chinese Hamster Ovary cell-line) transfected with pcDNA for human H1 receptor. Cells were dispensed into 96-well assay plates at a density of 25 000-30 000 cells per well and, following over night recovery, assayed for calcium response. Tests were performed using Fluo-4NW Calcium Assay Kit (Molecular Probes #F36206) and standard testing procedure for this kit. Cells were preincubated with test compound for 30 min, then endogenous agonist histamine was added. Fluorescence was measured by FLEXstation 3 (Molecular Devices). The apparent dissociation constants (Kb) were calculated by means of GraphPhad Prism 5 software using the modified Cheng Prusoff equation (Kb=IC50/(1+(A/EC50A)), where A=concentration of reference agonist in the assay and EC50A=EC50 value of the reference agonist). For each IC50 determination, agonist dose response curve is run in parallel with the antagonist, on the same experimental plate. Curves maximal and minimal values should be reached in assays and it is checked if Prism uses proper maximal and minimal values for curve fitting. The results are shown in Table 5.

TABLE 5
H₁ antagonistic equilibrium dissociation
constants determined by functional assay

| Compound of example | H₁ antagonist Kb [nM] |
|---|---|
| 1 | >5000.00 |
| 2 | 462.20 |
| 3 | >5000.00 |
| 4 | >5000.00 |
| 5 | 1225.00 |
| 6 | >1000.00 |
| 7 | >2000.00 |
| 8 | 19.45 |
| 9 | >5000.00 |

### Experiment 6: Alpha1 Functional Assay

The alpha-1 (α1) adrenergic receptor is coupled to the Gq heterotrimeric G-protein that activates phospholipase C and the phosphatidylinositol (PIP2) signaling pathway. The pathway starts with stimulation of the receptor and further activation of phospholipase C (PLC). Inositol-1,4,5-triphosphate (IP3), which is one of the products of PLC activity, binds to IP3 receptor in smooth ER. Opening of IP3R, which acts as a calcium ion channel, causes an increase in Ca²⁺ concentration in cytosol, which can be quantitatively measured using calcium-sensitive fluorescent dyes.

Alpha1 antagonism was measured in LNCaP (Human lymph node carcinoma of the prostate) cells expressing human alpha1 receptor. Cells were dispensed into 96-well assay plates at a density of 30 000 cells per well and, following over night recovery, assayed for calcium response. Tests were performed using Fluo-4NW Calcium Assay Kit (Molecular Probes #F36206) and standard testing procedure for this kit. Cells were preincubated with test compound for 30 min, then endogenous agonist noradrenaline was added. Fluorescence was measured by FLEXstation 3 (Molecular Devices). The apparent dissociation constants (Kb) were calculated by means of GraphPhad Prism 5 software using the modified Cheng Prusoff equation (Kb=IC50/(1+(A/EC50A)), where A=concentration of reference agonist in the assay and EC50A=EC50 value of the reference agonist). For each IC50 determination, agonist dose response curve is run in parallel with the antagonist, on the same experimental plate. Curves maximal and minimal values should be reached in assays, and it is checked if Prism uses proper maximal and minimal values for curve fitting. The results are shown in Table 6.

TABLE 6
Alpha1 antagonistic equilibrium dissociation
constants determined by functional assay

| Compound of example | Alpha1 antagonist Kb |
|---|---|
| 1 | 214.40 |
| 2 | >2000.00 |
| 3 | 83.84 |
| 4 | 203.00 |
| 5 | 171.40 |
| 6 | >2000.00 |
| 7 | 29.32 |
| 8 | 1601.00 |
| 9 | >2000.00 |

The compounds of formula I possess antagonist activity at the 5-HT₆ receptor. The present disclosure thus provides compounds for use as a medicament. Compounds for use in the treatment of disorder, condition, or disease mediated by the activity of 5-HT₆ receptors are also provided. Furthermore, a method for the treatment of disorder, condition, or disease mediated by 5-HT₆ receptor activity is provided. In said method an effective amount of at least one compound of formula I is administered to a mammal, such as a human, in need of such treatment. The use of the compounds of formula I for the manufacture of a medicament for the treatment of disorder, condition, or disease mediated by 5-HT₆ receptor activity is also provided.

In one embodiment of the invention the aforementioned disorder, condition, or disease mediated by 5-HT₆ receptor activity is cognitive memory impairment, such as Alzheimer's disease (AD), mild cognitive impairment (MCI), schizophrenia, Parkinson's disease (PD) or Huntington's disease (HD), or other dementias. The compounds of the present disclosure can be administered, for example, enterally, topically, or parenterally by means of any pharmaceutical formulation useful for said administration and comprising at least one active compound of formula I in pharmaceutically acceptable and effective amounts together with pharmaceutically acceptable diluents, carriers, and/or excipients known in the art. The manufacture of such pharmaceutical formulations is known in the art.

The therapeutic dose to be given to a subject in need of the treatment will vary depending on the compound being administered, the species, the age and the sex of the subject being treated, the particular condition being treated, as well as the route and method of administration, and may be determined by a person skilled in the art. A typical dosage for oral administration is from 10 ng/kg to 100 mg/kg per day and for parenteral administration from 1 ng/kg to 10 mg/kg for an adult mammal.

The compounds of the present disclosure are given to the subject as such or in combination with one or more other active ingredients, each in its own composition or some or all of the active ingredients combined in a single composition, and/or suitable pharmaceutical excipients. Suitable pharmaceutical excipients include conventionally used excipients and formulation aids, such as fillers, binders, disintegrating agents, lubricants, solvents, gel forming agents, emulsifiers, stabilizers, colorants, and/or preservatives.

The compounds of the present disclosure are formulated into dosage forms using commonly known pharmaceutical manufacturing methods. The dosage forms can be, for example, tablets, capsules, granules, suppositories, emulsions, suspensions, or solutions. Depending on the route of administration and the galenic form, the amount of the active ingredient in a formulation can typically vary between 0.01% and 100% by weight.

A person skilled in the art will appreciate that the embodiments described in the present disclosure can be modified without departing from the inventive concept. A person skilled in the art also understands that the present disclosure is not limited to the particular embodiments disclosed but is intended to also cover modifications of the embodiments that are within the scope of the present disclosure.

1. A compound of Formula I, 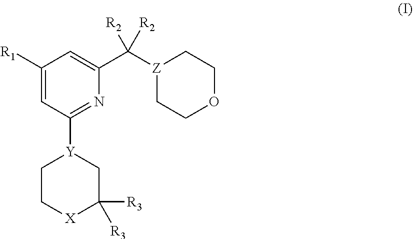
wherein
X is NR₄ or CR₅H;
Y is N or CH;
Z is CH or N;
R₁ is 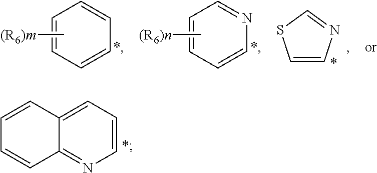
wherein the atom marked with the asterisk is bonded to the parent molecular moiety;
R₂ is, independently at each occurrence, H, (C₁-C₃)alkyl, or halogen;
or R₂ and R₂ both bonded to the same carbon atom form, together with the carbon atom to which they are bonded, a —(C═O) group;
R₃ is, independently at each occurrence, H, (C₁-C₃)alkyl, or halogen;
or R₃ and R₃ both bonded to the same carbon ring atom form, together with the carbon ring atom to which they are bonded, a —(C═O) group;
R₄ is H, (C₁-C₃)alkyl, or CF₃;
R₅ is N(R₇)₂;
R₆ is, independently at each occurrence, hydroxy, halogen, (C₁-C₃)alkyl, (C₁-C₃)alkoxy, CF₃, or (C₁-C₃)alkoxy(C₁-C₃)alkoxy;
R₇ is, independently at each occurrence, H or (C₁-C₃)alkyl;
m is 0, 1, 2 or 3; and
n is 0, 1 or 2
or a pharmaceutically acceptable salt or ester thereof.

2. The compound according to claim 1, wherein
X is NR₄ or CR₅H;
Y is N;
Z is CH;
R₁ is 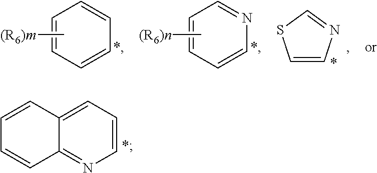
wherein the atom marked with the asterisk is bonded to the parent molecular moiety;
R₂ is, independently at each occurrence, H or (C₁-C₃)alkyl;
R₃ is, independently at each occurrence, H or (C₁-C₃)alkyl;
R₄ is H or (C₁-C₃)alkyl:
R₅ is N(R₇)₂;
R₆ is, independently at each occurrence, halogen or (C₁-C₃)alkoxy;
R₇ is H;
m is 1 or 2; and
n is 0.

3. The compound according to claim 1, wherein
X is NR₄;
Y is N;
Z is CH;
R₁ is 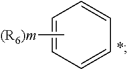
wherein the atom marked with the asterisk is bonded to the parent molecular moiety;
R₂ is, independently at each occurrence, H or (C₁-C₃)alkyl;
R₃ is, independently at each occurrence, H or (C₁-C₃)alkyl;
R₄ is H or (C₁-C₃)alkyl:
R₆ is halogen; and
m is 1.

4. The compound according to claim 1, wherein
X is NR₄;
Y is N;
Z is CH;
R₁ is 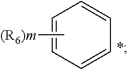
wherein the atom marked with the asterisk is bonded to the parent molecular moiety;
R₂ is, independently at each occurrence, H or (C₁-C₃)alkyl;
R₃ is, independently at each occurrence, H or (C₁-C₃)alkyl;
R₄ is H or (C₁-C₃)alkyl:
R₆ is (C₁-C₃)alkoxy; and
m is 1.

5. The compound according to claim 1, wherein the compound is 1-[4-(4-methoxyphenyl)-6-(oxan-4-ylmethyl)pyridin-2-yl]-4-methylpiperazine, 2-[2-(4-methylpiperazin-1-yl)-6-(oxan-4-ylmethyl)pyridin-4-yl]quinoline, 1-methyl-4-[6-(oxan-4-ylmethyl)-4-(pyridin-2-yl)pyridin-2-yl]piperazine, 1-methyl-4-[6-(oxan-4-ylmethyl)-4-(1,3-thiazol-4-yl)pyridin-2-yl]-piperazine, 1-[4-(2-fluoro-4-methoxyphenyl)-6-(oxan-4-ylmethyl)pyridin-2-yl]-4-methylpiperazine, 1-[4-(4-methoxyphenyl)-6-(oxan-4-ylmethyl)pyridin-2-yl]piperidin-4-amine, 1-[4-(4-methoxyphenyl)-6-[1-(oxan-4-yl)ethyl]pyridin-2-yl]-4-methylpiperazine, 1-[4-(4-methoxyphenyl)-6-(oxan-4-ylmethyl)pyridin-2-yl]-3,3-dimethylpiperazine, 1-[4-(4-methoxyphenyl)-6-(oxan-4-ylmethyl)pyridin-2-yl]piperazine, 1-[4-(4-methoxyphenyl)-6-(oxan-4-ylmethyl)pyridin-2-yl]-4-methyl-piperazine maleate, 1-[4-(4-methoxyphenyl)-6-(oxan-4-ylmethyl)pyridin-2-yl]-4-methyl-piperazine tartrate, 1-[4-(4-methoxyphenyl)-6-(oxan-4-ylmethyl)pyridin-2-yl]-4-methyl-piperazine citrate, or 1-[4-(4-methoxyphenyl)-6-(oxan-4-ylmethyl)pyridin-2-yl]-4-methyl-piperazine fumarate.

6. (canceled)

7. (canceled)

8. (canceled)

9. A method for the treatment of a disorder, condition, or disease mediated by 5-HT₆ receptor activity, which method comprises administering to a mammal in need of such treatment an effective amount of at least one compound according to claim 1.

10. The method according to claim 9, wherein the disorder, condition, or disease is cognitive memory impairment, such as Alzheimer's disease (AD), mild cognitive impairment (MCI), schizophrenia, Parkinson's disease (PD), or Huntington's disease (HD), or other dementia.

11. A pharmaceutical composition comprising as an active ingredient at least one compound according to claim 1 and a pharmaceutically acceptable carrier, diluent, and/or excipient.

12. The pharmaceutical composition according to claim 11, wherein the composition further comprises at least one other active ingredient.

13. The compound according to claim 2, wherein
X is NR₄;
Y is N;
Z is CH;
R₁ is 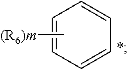
wherein the atom marked with the asterisk is bonded to the parent molecular moiety;
R₂ is, independently at each occurrence, H or (C₁-C₃)alkyl;
R₃ is, independently at each occurrence, H or (C₁-C₃)alkyl;
R₄ is H or (C₁-C₃)alkyl:
R₆ is halogen; and
m is 1.

14. The compound according to claim 2, wherein
X is NR₄;
Y is N;
Z is CH;
R₁ is 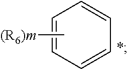
wherein the atom marked with the asterisk is bonded to the parent molecular moiety;
R₂ is, independently at each occurrence, H or (C₁-C₃)alkyl;
R₃ is, independently at each occurrence, H or (C₁-C₃)alkyl;
R₄ is H or (C₁-C₃)alkyl:
R₆ is (C₁-C₃)alkoxy; and
m is 1.

15. The method according to claim 9, wherein
X is NR₄ or CR₅H;
Y is N;
Z is CH;
R₁ is 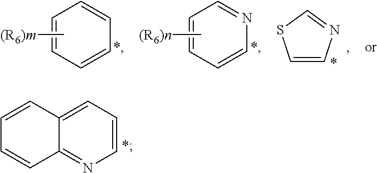
wherein the atom marked with the asterisk is bonded to the parent molecular moiety;
R₂ is, independently at each occurrence, H or (C₁-C₃)alkyl;
R₃ is, independently at each occurrence, H or (C₁-C₃)alkyl;
R₄ is H or (C₁-C₃)alkyl:
R₅ is N(R₇)₂;
R₆ is, independently at each occurrence, halogen or (C₁-C₃)alkoxy;
R₇ is H;
m is 1 or 2; and
n is 0.

16. The method according to claim 9, wherein
X is NR₄;
Y is N;
Z is CH;
R₁ is 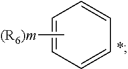
wherein the atom marked with the asterisk is bonded to the parent molecular moiety;
R₂ is, independently at each occurrence, H or (C₁-C₃)alkyl;
R₃ is, independently at each occurrence, H or (C₁-C₃)alkyl;
R₄ is H or (C₁-C₃)alkyl:
R₆ is halogen; and
m is 1.

17. The method according to claim 9, wherein
X is NR₄;
Y is N;
Z is CH;
R₁ is 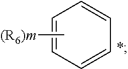
wherein the atom marked with the asterisk is bonded to the parent molecular moiety;
R₂ is, independently at each occurrence, H or (C₁-C₃)alkyl;
R₃ is, independently at each occurrence, H or (C₁-C₃)alkyl;
R₄ is H or (C₁-C₃)alkyl:
R₆ is (C₁-C₃)alkoxy; and
m is 1.

18. The method according to claim 9, wherein the compound is 1-[4-(4-methoxyphenyl)-6-(oxan-4-ylmethyl)pyridin-2-yl]-4-methylpiperazine, 2-[2-(4-methylpiperazin-1-yl)-6-(oxan-4-ylmethyl)pyridin-4-yl]quinoline, 1-methyl-4-[6-(oxan-4-ylmethyl)-4-(pyridin-2-yl)pyridin-2-yl]piperazine, 1-methyl-4-[6-(oxan-4-ylmethyl)-4-(1,3-thiazol-4-yl)pyridin-2-yl]-piperazine, 1-[4-(2-fluoro-4-methoxyphenyl)-6-(oxan-4-ylmethyl)pyridin-2-yl]-4-methylpiperazine, 1-[4-(4-methoxyphenyl)-6-(oxan-4-ylmethyl)pyridin-2-yl]piperidin-4-amine, 1-[4-(4-methoxyphenyl)-6-[1-(oxan-4-yl)ethyl]pyridin-2-yl]-4-methylpiperazine, 1-[4-(4-methoxyphenyl)-6-(oxan-4-ylmethyl)pyridin-2-yl]-3,3-dimethylpiperazine, 1-[4-(4-methoxyphenyl)-6-(oxan-4-ylmethyl)pyridin-2-yl]piperazine, 1-[4-(4-methoxyphenyl)-6-(oxan-4-ylmethyl)pyridin-2-yl]-4-methyl-piperazine maleate, 1-[4-(4-methoxyphenyl)-6-(oxan-4-ylmethyl)pyridin-2-yl]-4-methyl-piperazine tartrate, 1-[4-(4-methoxyphenyl)-6-(oxan-4-ylmethyl)pyridin-2-yl]-4-methyl-piperazine citrate, or 1-[4-(4-methoxyphenyl)-6-(oxan-4-ylmethyl)pyridin-2-yl]-4-methyl-piperazine fumarate.

19. The pharmaceutical composition according to claim 11, wherein
X is NR₄ or CR₅H;
Y is N;
Z is CH;
R₁ is 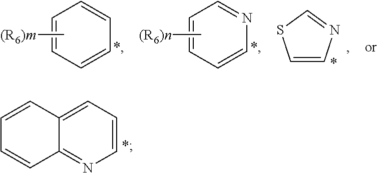
wherein the atom marked with the asterisk is bonded to the parent molecular moiety;
R₂ is, independently at each occurrence, H or (C₁-C₃)alkyl;
R₃ is, independently at each occurrence, H or (C₁-C₃)alkyl;
R₄ is H or (C₁-C₃)alkyl:
R₅ is N(R₇)₂;
R₆ is, independently at each occurrence, halogen or (C₁-C₃)alkoxy;
R₇ is H;
m is 1 or 2; and
n is 0.

20. The pharmaceutical composition according to claim 11, wherein
X is NR₄;
Y is N;
Z is CH;
R₁ is 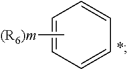
wherein the atom marked with the asterisk is bonded to the parent molecular moiety;
R₂ is, independently at each occurrence, H or (C₁-C₃)alkyl;
R₃ is, independently at each occurrence, H or (C₁-C₃)alkyl;
R₄ is H or (C₁-C₃)alkyl:
R₆ is halogen; and
m is 1.

21. The pharmaceutical composition according to claim 11, wherein
X is NR₄;
Y is N;
Z is CH;
R₁ is 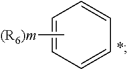
wherein the atom marked with the asterisk is bonded to the parent molecular moiety;
R₂ is, independently at each occurrence, H or (C₁-C₃)alkyl;
R₃ is, independently at each occurrence, H or (C₁-C₃)alkyl;
R₄ is H or (C₁-C₃)alkyl:
R₆ is (C₁-C₃)alkoxy; and
m is 1.

22. The pharmaceutical composition according to claim 11, wherein the compound is 1-[4-(4-methoxyphenyl)-6-(oxan-4-ylmethyl)pyridin-2-yl]-4-methylpiperazine, 2-[2-(4-methylpiperazin-1-yl)-6-(oxan-4-ylmethyl)pyridin-4-yl]quinoline, 1-methyl-4-[6-(oxan-4-ylmethyl)-4-(pyridin-2-yl)pyridin-2-yl]piperazine, 1-methyl-4-[6-(oxan-4-ylmethyl)-4-(1,3-thiazol-4-yl)pyridin-2-yl]-piperazine, 1-[4-(2-fluoro-4-methoxyphenyl)-6-(oxan-4-ylmethyl)pyridin-2-yl]-4-methylpiperazine, 1-[4-(4-methoxyphenyl)-6-(oxan-4-ylmethyl)pyridin-2-yl]piperidin-4-amine, 1-[4-(4-methoxyphenyl)-6-[1-(oxan-4-yl)ethyl]pyridin-2-yl]-4-methylpiperazine, 1-[4-(4-methoxyphenyl)-6-(oxan-4-ylmethyl)pyridin-2-yl]-3,3-dimethylpiperazine, 1-[4-(4-methoxyphenyl)-6-(oxan-4-ylmethyl)pyridin-2-yl]piperazine, 1-[4-(4-methoxyphenyl)-6-(oxan-4-ylmethyl)pyridin-2-yl]-4-methyl-piperazine maleate, 1-[4-(4-methoxyphenyl)-6-(oxan-4-ylmethyl)pyridin-2-yl]-4-methyl-piperazine tartrate, 1-[4-(4-methoxyphenyl)-6-(oxan-4-ylmethyl)pyridin-2-yl]-4-methyl-piperazine citrate, or 1-[4-(4-methoxyphenyl)-6-(oxan-4-ylmethyl)pyridin-2-yl]-4-methyl-piperazine fumarate.
